(19) United States
(12) Patent Application Publication
(10) Pub. No.: US 2024/0091255 A1
(43) Pub. Date: Mar. 21, 2024

(54) TREATMENT OF ASTIGMATISM

(71) Applicant: iVeena Delivery Systems, Inc., Salt Lake City, UT (US)

(72) Inventors: Balamurali Ambati, Eugene, OR (US); Sarah A. Molokhia, Sandy, UT (US)

(21) Appl. No.: 18/274,647

(22) PCT Filed: Jan. 28, 2022
(86) PCT No.: PCT/US22/14198

§ 371 Date: Jul. 27, 2023

Related U.S. Application Data
(60) Provisional application No. 63/144,237, filed on Feb. 1, 2021.

(51) Int. Cl. A61K 33/34; A61K 9/00; A61P 27/10

(52) U.S. Cl. CPC A61K 33/34; A61K 9/0048; A61P 27/10

(57) ABSTRACT

The present invention provides methods of treating corneal astigmatism comprising administering composition comprising a copper-containing agent to an eye of a subject in need thereof.

### RELATED APPLICATION

This application claims the benefit of U.S. Provisional Application No. 63/144,237 filed on Feb. 1, 2021, the contents of which are fully incorporated by reference herein.

### BACKGROUND

Astigmatism is an eye disorder characterized by a refractive error in which the eye does not focus light evenly on the retina. The underlying defect of astigmatism is an irregular curvature of the cornea or abnormalities in the lens of the eye. Astigmatism is very common, with an estimated 30-60% of adults in the US and Europe estimated to have astigmatism. Though symptoms may be mild, in more severe cases, astigmatism can cause distorted or blurred vision, eye strain, headaches, poor night vision, fatigue, and squinting. Astigmatism is often treated with corrective lenses, or in some cases, with refractive surgery. Further non-invasive methods of treating astigmatism are needed.

### SUMMARY OF THE INVENTION

The present invention relates to methods of treating corneal astigmatism, comprising administering a composition comprising a copper-containing agent to an eye of a subject in need thereof.

### DETAILED DESCRIPTION OF THE INVENTION

Methods of the Invention

Astigmatism is a common eye disorder characterized by a refractive error in which the eye does not focus light evenly on the retina. Though some astigmatism is mild, in severe cases, astigmatism can cause distorted or blurred vision, eye strain, headaches, poor night vision, fatigue, and squinting. As described herein, topical application of a copper compound to the eye is effective to treat astigmatism. This discovery was particularly unexpected since no pharmacologic agents are known to reduce corneal astigmatism.

Accordingly, the present invention is directed to a method of treating corneal astigmatism, comprising administering a composition comprising a copper-containing agent to an eye of a subject in need thereof.

In certain embodiments, an ophthalmic composition or dosage form comprising the copper-containing agent is used in the methods of the invention. The ophthalmic composition or dosage form can include an amount of a copper-containing agent effective to treat astigmatism. The composition or dosage form can further include a pharmaceutically acceptable carrier. In some embodiments, the dosage form can be an ophthalmic composition formulated as a topical eye drop. Such compositions may be carried in a container adapted to dispense the composition in a drop-wise manner, e.g., at a drop volume of from about 5 μl to about 100 μl. In some embodiments, the ophthalmic composition can be a sustained release composition that is formulated to release the copper-containing agent over a prolonged period of time. The method of the invention can include administering a therapeutically effective amount of a composition or dosage form, as described herein, during a treatment period.

In certain embodiments, the copper-containing agent is a copper salt. In certain embodiments, the copper-containing agent is selected from: copper sulfate, copper carbonate, copper acetate, copper chloride, copper gluconate, copper bromide, copper fluoride, copper nitrate, copper iodide, copper perchlorate, copper periodate, copper perbromate, copper permanganate, hemocyanin, copper molybdate, copper thiocyanate, copper tartrate, copper tetrafluoroborate, copper selenide, copper pyrophosphate, GHK-copper, tetra-amine copper sulfate, copper-histidine, and copper-glycinate. In further embodiments, the copper containing agent is selected from: copper sulfate, copper acetate, copper chloride, copper gluconate, copper bromide, copper fluoride, copper nitrate, copper perchlorate, and copper tartrate, hydrates thereof, or combinations thereof. In certain preferred embodiments, the copper-containing agent is copper sulfate. In manufacturing the ophthalmic compositions described herein, the copper sulfate that is used in the manufacture may be anhydrous or a hydrate (e.g., copper (II) sulfate pentahydrate).

Ophthalmologic compositions of a copper-containing agent can be characterized based upon the amount of copper in the composition, which may be conveyed by any suitable measure of concentration, e.g., molality, molarity, or as a wt % of the copper-containing agent in the composition.

In some embodiments, the weight percentages provided herein are calculated based on the percent weight of anhydrous copper (II) sulfate in the ophthalmologic composition, e.g., as a way of normalizing copper content independent of the weight of any associated counterions, complexes, or ligands. Thus, where an alternative copper-containing agent is employed, the weight percentages can be converted accordingly based on the molecule weight of the copper agent.

For example, the copper-containing agent can be present in an amount from about 0.00001 wt % or about 0.0001 wt % to about 5 wt %, 10 wt %, or 15 wt %. In some embodiments, the copper-containing agent can be present from about 0.0001 wt % to about 5 wt %, from about 0.0001 wt % to about 4 wt %, from about 0.0001 wt % to about 3 wt %, from about 0.0001 wt % to about 2 wt %, from about 0.0001 wt % to about 1 wt %, 0.0001 wt % to about 0.75 wt %, from about 0.0001 wt % to about 0.5 wt %, from about 0.0001 wt % to about 0.25 wt %, from about 0.0001 wt % to about 0.1 wt %, from about 0.0001 wt % to about 0.075 wt %, from about 0.0001 wt % to about 0.05 wt %, from about 0.0001 wt % to about 0.025 wt %, or from about 0.0001 wt % to about 0.02 wt %.

Alternatively, in some embodiments, the copper-containing agent can be present from about 0.0005 wt % to about 5 wt %, from about 0.0005 wt % to about 4 wt %, from about 0.0005 wt % to about 3 wt %, from about 0.0005 wt % to about 2 wt %, from about 0.0005 wt % to about 1 wt %, 0.0005 wt % to about 0.75 wt %, from about 0.0005 wt % to about 0.5 wt %, from about 0.0005 wt % to about 0.25 wt %, from about 0.0005 wt % to about 0.1 wt %, from about 0.0005 wt % to about 0.075 wt %, from about 0.0005 wt % to about 0.05 wt %, from about 0.0005 wt % to about 0.025 wt %, or from about 0.0005 wt % to about 0.02 wt %.

Alternatively, in some embodiments, the copper-containing agent can be present from about 0.001 wt % to about 5 wt %, from about 0.001 wt % to about 4 wt %, from about 0.001 wt % to about 3 wt %, from about 0.001 wt % to about 2 wt %, from about 0.001 wt % to about 1 wt %, 0.001 wt % to about 0.75 wt %, from about 0.001 wt % to about 0.5 wt %, from about 0.001 wt % to about 0.25 wt %, from about 0.001 wt % to about 0.1 wt %, from about 0.001 wt % to about 0.075 wt %, from about 0.001 wt % to about 0.05 wt %, from about 0.001 wt % to about 0.025 wt %, or from about 0.001 wt % to about 0.02 wt %.

Alternatively, in some embodiments, the copper-containing agent can be present from about 0.003 wt % to about 5 wt %, from about 0.003 wt % to about 4 wt %, from about 0.003 wt % to about 3 wt %, from about 0.003 wt % to about 2 wt %, from about 0.003 wt % to about 1 wt %, 0.003 wt % to about 0.75 wt %, from about 0.003 wt % to about 0.5 wt %, from about 0.003 wt % to about 0.25 wt %, from about 0.003 wt % to about 0.1 wt %, from about 0.003 wt % to about 0.075 wt %, from about 0.003 wt % to about 0.05 wt %, from about 0.003 wt % to about 0.025 wt %, or from about 0.003 wt % to about 0.02 wt %.

Alternatively, in some embodiments, the copper-containing agent can be present from about 0.005 wt % to about 5 wt %, from about 0.005 wt % to about 4 wt %, from about 0.005 wt % to about 3 wt %, from about 0.005 wt % to about 2 wt %, from about 0.005 wt % to about 1 wt %, 0.005 wt % to about 0.75 wt %, from about 0.005 wt % to about 0.5 wt %, from about 0.005 wt % to about 0.25 wt %, from about 0.005 wt % to about 0.1 wt %, from about 0.005 wt % to about 0.075 wt %, from about 0.005 wt % to about 0.05 wt %, from about 0.005 wt % to about 0.025 wt %, or from about 0.005 wt % to about 0.02 wt %.

In further embodiments, the copper-containing agent can be present from about 0.05 wt % to about 15 wt %, from about 0.01 wt % to about 10 wt %, or from about 0.005 wt % to about 5 wt %. In other embodiments, the copper-containing agent can be present in an amount from about 0.00001 wt % to about 0.0001 wt %, from about 0.0001 wt % to about 0.0005 wt %, from about 0.0001 wt % to about 0.0002 wt %, from about 0.0002 wt % to about 0.0003 wt %, or from about 0.0003 wt % to about 0.0004 wt %. In yet other embodiments, the copper-containing agent can be present in an amount from about 0.001 wt % to about 0.01 wt % or about 0.003 wt % to about 0.008 wt %. In yet other embodiments, the copper-containing agent can be present in an amount from about 0.01 wt % to about 0.1 wt %, or from about 0.03 wt % to about 0.08 wt %.

The amount of copper in the composition can alternatively be expressed as weight per volume (e.g., mg/mL). In some embodiments, the weight per volume measurements provided herein are calculated based on the weight of total copper per volume unit in the ophthalmologic composition. For example, an amount of 0.0025 mg/ml of copper (II) sulfate pentahydrate provides the composition with a copper content of about 0.000636 mg/ml copper. This is because the atomic weight of copper (II) sulfate pentahydrate is about 249.677 g/mol, but only about 63.5 g/mol or about 25% of the agent is copper itself. As an alternative example, an amount of 0.0018 mg/ml of copper (II) acetate, anhydrous, provides the composition with a copper content of about 0.00063 mg/ml. Thus, in some embodiments, the concentration of the copper-containing agent can be determined based on the total copper content provided by the copper-containing agent rather than the amount of copper-containing agent itself.

According, in some embodiments, the composition comprises the copper-containing agent in an amount from about 0.0001 mg/mL or about 0.0005 mg/mL to about 5 mg/mL or about 50 mg/mL. In some embodiments, the composition comprises the copper-containing agent in an amount from about 0.001 mg/mL to about 50 mg/mL, from about 0.001 mg/mL to about 40 mg/mL, from about 0.001 mg/mL to about 30 mg/mL, from about 0.001 mg/mL to about 20 mg/mL, from about 0.001 mg/mL to about 10 mg/mL, 0.001 mg/mL to about 7.5 mg/mL, from about 0.001 mg/mL to about 5 mg/mL, from about 0.001 mg/mL to about 2.5 mg/mL, from about 0.001 mg/mL to about 1 mg/mL, from about 0.001 mg/mL to about 0.75 mg/mL, from about 0.001 mg/mL to about 0.5 mg/mL, from about 0.001 mg/mL to about 0.25 mg/mL, or from about 0.001 mg/mL to about 0.2 mg/mL.

Alternatively, in some embodiments, the copper-containing agent can be present from about 0.005 mg/mL to about 50 mg/mL, from about 0.005 mg/mL to about 40 mg/mL, from about 0.005 mg/mL to about 30 mg/mL, from about 0.005 mg/mL to about 20 mg/mL, from about 0.005 mg/mL to about 10 mg/mL, 0.005 mg/mL to about 7.5 mg/mL, from about 0.005 mg/mL to about 5 mg/mL, from about 0.005 mg/mL to about 2.5 mg/mL, from about 0.005 mg/mL to about 1 mg/mL, from about 0.005 mg/mL to about 0.75 mg/mL, from about 0.005 mg/mL to about 0.5 mg/mL, from about 0.005 mg/mL to about 0.25 mg/mL, or from about 0.005 mg/mL to about 0.2 mg/mL.

Alternatively, in some embodiments, the copper-containing agent can be present from about 0.01 mg/mL to about 50 mg/mL, from about 0.01 mg/mL to about 40 mg/mL, from about 0.01 mg/mL to about 30 mg/mL, from about 0.01 mg/mL to about 20 mg/mL, from about 0.01 mg/mL to about 10 mg/mL, 0.01 mg/mL to about 7.5 mg/mL, from about 0.01 mg/mL to about 5 mg/mL, from about 0.01 mg/mL to about 2.5 mg/mL, from about 0.01 mg/mL to about 1 mg/mL, from about 0.01 mg/mL to about 0.75 mg/mL, from about 0.01 mg/mL to about 0.5 mg/mL, from about 0.01 mg/mL to about 0.25 mg/mL, or from about 0.01 mg/mL to about 0.2 mg/mL.

Alternatively, in some embodiments, the copper-containing agent can be present from about 0.03 mg/mL to about 50 mg/mL, from about 0.03 mg/mL to about 40 mg/mL, from about 0.03 mg/mL to about 30 mg/mL, from about 0.03 mg/mL to about 20 mg/mL, from about 0.03 mg/mL to about 10 mg/mL, 0.03 mg/mL to about 7.5 mg/mL, from about 0.03 mg/mL to about 5 mg/mL, from about 0.03 mg/mL to about 2.5 mg/mL, from about 0.03 mg/mL to about 1 mg/mL, from about 0.03 mg/mL to about 0.75 mg/mL, from about 0.03 mg/mL to about 0.5 mg/mL, from about 0.03 mg/mL to about 0.25 mg/mL, or from about 0.03 mg/mL to about 0.2 mg/mL.

Alternatively, in some embodiments, the copper-containing agent can be present from about 0.05 mg/mL to about 50 mg/mL, from about 0.05 mg/mL to about 40 mg/mL, from about 0.05 mg/mL to about 30 mg/mL, from about 0.05 mg/mL to about 20 mg/mL, from about 0.05 mg/mL to about 10 mg/mL, 0.05 mg/mL to about 7.5 mg/mL, from about 0.05 mg/mL to about 5 mg/mL, from about 0.05 mg/mL to about 2.5 mg/mL, from about 0.05 mg/mL to about 1 mg/mL, from about 0.05 mg/mL to about 0.75 mg/mL, from about 0.05 mg/mL to about 0.5 mg/mL, from about 0.05 mg/mL to about 0.4 mg/mL, from about 0.05 mg/mL to about 0.3 mg/mL, from about 0.05 mg/mL to about 0.25 mg/mL, from about 0.05 mg/mL to about 0.2 mg/mL, or from about 0.05 mg/mL to about 0.2 mg/mL.

Alternatively, in some embodiments, the copper-containing agent can be present from about 0.075 mg/mL to about 50 mg/mL, from about 0.075 mg/mL to about 40 mg/mL, from about 0.075 mg/mL to about 30 mg/mL, from about 0.075 mg/mL to about 20 mg/mL, from about 0.075 mg/mL to about 10 mg/mL, 0.075 mg/mL to about 7.5 mg/mL, from about 0.075 mg/mL to about 5 mg/mL, from about 0.075 mg/mL to about 2.5 mg/mL, from about 0.075 mg/mL to about 1 mg/mL, from about 0.075 mg/mL to about 0.75 mg/mL, from about 0.075 mg/mL to about 0.5 mg/mL, from about 0.075 mg/mL to about 0.4 mg/mL, from about 0.075 mg/mL to about 0.3 mg/mL, from about 0.075 mg/mL to about 0.25 mg/mL, from about 0.075 mg/mL to about 0.2 mg/mL, or from about 0.075 mg/mL to about 0.2 mg/mL.

Alternatively still, in some embodiments, the copper-containing agent can be present from about 0.1 mg/mL to about 50 mg/mL, from about 0.1 mg/mL to about 40 mg/mL, from about 0.1 mg/mL to about 30 mg/mL, from about 0.1 mg/mL to about 20 mg/mL, from about 0.1 mg/mL to about 10 mg/mL, 0.1 mg/mL to about 7.5 mg/mL, from about 0.1 mg/mL to about 5 mg/mL, from about 0.1 mg/mL to about 2.5 mg/mL, from about 0.1 mg/mL to about 1 mg/mL, from about 0.1 mg/mL to about 0.75 mg/mL, from about 0.1 mg/mL to about 0.5 mg/mL, from about 0.1 mg/mL to about 0.4 mg/mL, from about 0.1 mg/mL to about 0.3 mg/mL, from about 0.1 mg/mL to about 0.25 mg/mL, or from about 0.1 mg/mL to about 0.2 mg/mL.

However, a particular amount of copper-containing agent in the composition does not necessarily mean that all of the copper content will be bioavailable upon administration or will become bioavailable at the same rate. The bioavailability of the copper can vary to some extent from one copper-containing component to another. Additionally, the bioavailability of the copper can be affected by pH, viscosity, solubility, and other compositional factors. For a given patient, an appropriate dose of the copper-containing agent can be determined based on the type of delivery vehicle, the type of copper-containing agent, the desired delivery duration, etc. Thus, the appropriate dose of a copper-containing agent can also be adjusted based on the bioavailability of copper with respect to a particular copper carrier, pH, formulation, or the like. Further, the release rate of the copper content from a particular dosage form can be adjusted based on the particular copper-containing agent employed in the dosage form. For example, in some cases a less soluble copper-containing agent (e.g., copper fluoride, copper hydroxide, copper carbonate, for example) can be used to prolong the release of the copper-containing agent from the composition. In some further embodiments, the release rate can additionally or alternatively be controlled via a particular pharmaceutical carrier or formulation type.

In some embodiments, the copper-containing agent can be administered with a second active or therapeutic agent, such as an additional crosslinking agent. A second active agent can operate through alternative mechanisms of action that work in concert with the crosslinking induced by the copper-containing agent and/or another crosslinking agent. For example, second active agents can reduce axial elongation, reduce accommodation (i.e. the process by which the eye changes optical power to maintain a clear focus on an image as its distance varies), the like, or a combination thereof. Such additional agents can include riboflavin, rose bengal, hydroxylysine, a calcium-containing agent, a magnesium-containing agent, a silver-containing agent, an aluminum-containing agent, a zinc-containing agent, iron-containing agent, acai extract, decorin, biglycan, keratocan, lumican, mimican, fibromodulin, type VI collagen, type X collagen, type XII collagen, type XIV collagen, atropine, homatropine, cyclopentolate, pirenzepine, 7-methylxanthanine, the like, or combinations thereof. In some embodiments, the additional or second active agent can include atropine, homatropine, cyclopentolate, pirenzepine, 7-methylxanthanine, the like, or a combination thereof. In certain embodiments, the second active agent can include atropine. In some additional embodiments, the second active agent can include homatropine. In still additional embodiments, the second active agent can include cyclopentolate. In further embodiments, the second active agent can include pirenzepine. In still further embodiments, the second active agent can include 7-methylxanthanine. The second active agent can generally be present in an amount from about 0.001 wt % to about 0.1 wt %. In other embodiments, the second active agent can be present in an amount from about 0.005 wt % to about 0.05 wt %, or from about 0.007 wt % to about 0.02 wt %.

In other embodiments, the composition does not comprise an additional active ingredient, i.e., the copper-containing agent is the sole active ingredient of the composition. In some embodiments, the composition does not comprise an anti-inflammatory agent, an antihistamine, a vasoconstrictor, an antibiotic, an analgesic, or a steroid.

In certain embodiments, the composition does not comprise an amine. In some embodiments, the composition does not comprise an amine-copper complex, e.g., a coordination complex in which a copper cation is complexed to one or more amine ligands.

The copper-containing agent can be provided in a pharmaceutically acceptable carrier. The pharmaceutically acceptable carrier can be formulated in a variety of ways to deliver the copper-containing agent. Non-limiting examples can include solutions, suspensions, emulsions, gels, hydrogels, thermo-responsive gels, formulation for subconjunctival injection, formulation for sub-tenon's injection, depots, films, sustained-delivery matrixes, contact lenses, pledgets, punctal plugs, gellating suspensions, the like, or a combination thereof. The composition can be formulated for passive delivery to the eye. Alternatively, the composition can be formulated for active delivery to the eye, such as iontophoresis, electroporation, sonoporation, etc. In certain embodiments, the formulation can be an ophthalmic drop. In some embodiments, the composition can be formulated as a copper-eluting contact lens, such as a soft lens, a toric lens, a hard lens, a scleral lens, the like, or combination thereof. The contact lens can be a daily disposable lens or extended use lens (e.g., from 2 days-use to 2 week-use lenses or longer). In some embodiments, the composition can be formulated as a sustained-delivery matrix for placement in contact with an ocular surface, such as in a cul-de-sac, conjunctiva, tenon's capsule or sub-tenon's space, etc. In some embodiments, the composition can be formulated as a biodegradable device, such as a lens, film, capsule, punctal plug, the like, or a combination thereof. The biodegradable device can be configured to biodegrade at a rate of from about 1 week to about 6 months, or from about 2 weeks to about 4 months, or from about 1 month to about 2 months.

The composition comprising the copper-containing agent can further comprise an excipient selected from: a tonicity agent, a solubilizing agent, a thickener, a polymer, a buffer, a pH adjuster, a preservative, and water. In certain such embodiments, the composition comprises two or more of said excipients.

Non-limiting examples of solubilizing agents can include phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, BSS Plus, Ringer's lactate solution, normal saline (i.e. 0.9% saline), ½ normal saline, the like, or combinations thereof. Solubilizing agents can be present in the pharmaceutically acceptable carrier in various amounts depending on the particular formulation, method of treatment, etc.

Non-limiting examples of tonicity agents can include the solubilizing agents previously listed, as well as sodium chloride, potassium chloride, calcium chloride, magnesium chloride, mannitol, sorbitol, dextrose, glycerin, propylene glycol, ethanol, trehalose, the like, or combinations thereof. The tonicity agent can be used to provide an appropriate tonicity of the formulation. In certain embodiments, the tonicity of the formulation is from about 200 to about 800 milliosmoles/liter (mOsm/L). In other embodiments, the tonicity of the formulation can be from about 200 mOsm/L to about 700 mOsm/L. In other embodiments, the tonicity of the formulation can be from about 200 mOsm/L to about 650 mOsm/L. In yet other embodiments, the tonicity of the formulation can be from about 200 mOsm/L to about 600 mOsm/L. In other embodiments, the tonicity of the formulation can be from about 250 mOsm/L to about 700 mOsm/L. In still other embodiments, the tonicity of the formulation can be from about 250 mOsm/L to about 650 mOsm/L, from about 250 mOsm/L to about 600 mOsm/L, from about 270 mOsm/L to about 700 mOsm/L, from about 270 mOsm/L to about 650 mOsm/L, or from about 270 mOsm/L to about 600 mOsm/L. Tonicity agents can be present in the pharmaceutically acceptable carrier in various amounts depending on the particular formulation, method of treatment, etc.

Non-limiting examples of pH adjusters can include a number of acids, bases, and combinations thereof, such as hydrochloric acid, phosphoric acid, citric acid, sodium hydroxide, potassium hydroxide, calcium hydroxide, and the like. The pH adjusters can be used to provide an appropriate pH for the formulation. In certain embodiments, the pH of the composition can be from about 5.0 to about 8.5. In certain embodiments, the pH can be from about 5.0 to about 8.0. Alternatively, the pH can be about 5.2 to about 8.0, about 5.3 to about 8.0, about 5.4 to about 7.9, or about 5.5 to about 7.8. pH adjusters can be present in the pharmaceutically acceptable carrier in various amounts depending on the particular formulation, method of treatment, etc.

Non-limiting examples of thickeners can include glycerol, propylene glycol, polyethylene glycol, polyvinyl alcohol, cellulose derivatives (such as methyl cellulose, carboxymethyl cellulose, hydroxypropyl cellulose, and the like) ethylvinyl alcohol, hyaluronic acid, the like, or combinations thereof. Thickeners can be present in the pharmaceutically acceptable carrier in various amounts depending on the particular formulation, method of treatment, etc.

Non-limiting examples of polymers that can be used to prepare a polymer matrix for a film, contact lens or the like, can include biodegradable or non-biodegradable polymers. Non-limiting examples of polymers or polymer combinations can include poly(methylmethacrylate), polyorthoesters, hydroxyethylmethacrylate, polysiloxanes, poly(lactic-co-glycolic acid) (different ratios of lactic to glycolide content and end groups such as acid or ester termination), polyvinyl alcohol, polyvinyl acetate, ethylene vinyl acetate, polyethylene glycol, polylactic acid, polyglycolic acid, hydroxypropyl methylcellulose, hydroxypropylcellulose, carboxymethylcellulose, croscarmellose, polycaprolactone, hyaluronic acid, albumin, sodium chloride block copolymers thereof, salts thereof, the like, or combinations thereof. Specific copolymers such as polylactic-polyglycolic acid block copolymers (PLGA), polyglycolic acid-polyvinyl alcohol block copolymers (PGA/PVA), hydroxypropylmethylcellulose (HPMC), polycaprolactone-polyethylene glycol block copolymers, croscarmellose, and the like can be particularly effective for biodegradable matrixes, where desirable.

In certain embodiments, the composition can include thermo-responsive polymers. Non-limiting examples of thermo-responsive polymers can include poly(N-isopropyl acrylamide), poly [2-(dimethylamino)ethylmethacrylate], hydroxypropylcellulose, poly(vinyl caprolactam), polyvinyl methyl ether, polyethylene oxide, polyhydroxyethylmethacrylate, ABCBA-type pentablock polymers, chitosan, the like, or combinations thereof. Such thermo-responsive polymers can bind or can be functionalized to bind a particular copper-containing agent within a range of temperatures and release the copper-containing agent upon changing the temperature of the surrounding environment, such as placing the composition in contact with the eye, applying a heat source to the eye after administration of the composition, or the like.

Non-limiting examples of preservatives can include benzalkonium chloride (BAK), cetrimonium, sodium perborate, ethylenediaminetetraaceticacid (EDTA) and its various salt forms, chlorobutanol, and the like. Preservatives can be present in the pharmaceutically acceptable carrier in various amounts depending on the particular formulation, method of treatment, etc.

In certain preferred embodiments, the composition is administered topically. Compositions for topical administration to the eye include ophthalmic compositions. The composition for topical administration may be formulated as an ointment, gel, thin film or eye drop composition.

In particularly preferred embodiments, the composition can be formulated as an eye drop, e.g., wherein the pharmaceutically acceptable carrier can include PBS, BSS, or other suitable solubility or tonicity agent. For example, the composition can be formulated as an ophthalmic drop and the pharmaceutically acceptable carrier can include artificial tears (e.g. Refresh Tears®, Genteal®, Oasis Tears®, or the like). Alternatively, the composition can be formulated as a thin film, ointment, gellating suspension, punctal plug, or contact lens.

The ophthalmic composition can be used as an ophthalmic dosage form to administer a therapeutically effective dose of the copper-containing agent. In some embodiments, an amount from about 1 μl to about 500 μl of the composition is administered per administration event. In yet other embodiments, an amount from about 1 μl to about 250 μl, about 1 μl to about 200 μl, about 1 μl to about 100 μl, about 2 μl to about 250 μl, about 2 μl to about 200 μl, about 2 μl to about 100 μl, about 3 μl to about 250 μl, about 3 μl to about 200 μl, about 3 μl to about 100 μl, about 4 μl to about 250 μl, about 4 μl to about 200 μl about 4 μl to about 100 μl, or about 5 μl to about 250 μl, about 5 μl to about 200 μl, or about 5 μl to about 100 μl is administered per administration event. In the case of eyedrops, this volume is preferably administered in, for example, 1-3 drops of the ophthalmic composition.

In some embodiments, the ophthalmic composition can be formulated to biodegrade to otherwise provide controlled or sustained release of the copper-containing agent over a predetermined period of time. In yet other embodiments, the ophthalmic dosage form can be formulated to release the copper-containing agent from a non-biodegradable matrix in a controlled or sustained manner. In certain such embodiments, the dosage form can be formulated to release the copper-containing agent over a period of hours, days, or weeks, as desired. In some specific embodiments, the composition can be formulated to deliver from about 0.005 mcg of copper to about 250 mcg of copper per week. In yet other embodiments, the dosage form can be formulated to deliver from about 0.008 mcg to about 200 mcg per week, about 0.01 mcg to about 150 mcg per week, or about 0.1 mcg to about 100 mcg per week. In certain embodiments, the ophthalmic composition can be formulated to have zero-order drug release kinetics.

In some embodiments, the dosage form can be held in or stored in a container as a pre-mixed composition that is ready to administer without further dilution or preparation. In some embodiments, a single container can hold a volume or amount of the composition that is adequate for a single dose, but less than an amount that is adequate for a plurality of doses. In yet other embodiments, a single container can hold a volume or amount of the composition that is adequate for multiple doses.

A number of suitable containers can be used. The container can be, for example, an amber-colored container. In some embodiments, the container can be made of glass, polypropylene, polyethylene, polycarbonate, polyvinylchloride, the like, or a combination thereof. In some embodiments, the container can have a volume of from about 0.5 ml to about 50 ml. In other embodiments, the container can have a volume of from about 1 ml to about 30 ml, about 5 ml to about 20 ml, or about 3 ml to about 15 ml. In other embodiments, the container can hold a single dose of the dosage form. Alternatively, the container can hold a plurality of doses of the dosage form. The container can be, for example, a vial, a bottle, a blister pack, a sachet, or the like.

In some embodiments, about 0.005 mg to about 10 mg of the copper-containing agent can be included in the container. In yet other embodiments, about 0.01 mg to about 5 mg of the copper-containing agent can be included in the container. In some embodiments, about 0.001 mg to about 5 mg of copper can be included in the container. In some embodiments, about 0.005 mg to about 2 mg of copper can be included in the container.

In certain embodiments, the dosage form can be a topical ophthalmic dosage form that is formulated as an eye drop and carried in a container adapted to dispense the composition in a drop-wise manner at a drop volume of from about 5 μl to about 100 μl. Alternatively, the container can be adapted to dispense the ophthalmic composition at a drop volume of from about 1 μl to about 250 μl, about 1 μl to about 200 μl, about 1 μl to about 100 μl, about 2 μl to about 250 μl, about 2 μl to about 200 μl, about 2 μl to about 100 μl, about 3 μl to about 250 μl, about 3 μl to about 200 μl, about 3 μl to about 100 μl, about 4 μl to about 250 μl, about 4 μl to about 200 μl, about 4 μl to about 100 μl, or about 5 μl to about 250 μl, about 5 μl to about 200 μl, or about 5 μl to about 100 μl.

Where the composition is formulated as an eye drop, the container can include a fitted nozzle or tip from which the composition can be dispensed. As such, the container can typically be, at least partially, flexible or collapsible to dispense the composition. However, in some such cases, after the composition is dispensed, air can be sucked back into the container, which can contaminate the composition. Accordingly, the nozzle or tip can include a valve mechanism, filter, the like, or combination thereof to prevent or minimize introduction of bacteria and other contaminants into the container.

In certain embodiments, a container or dosage form can include or be accompanied by an administration mechanism, such as a syringe, a dropper, or other mechanism. In some embodiments, packaging can be configured to provide the composition, container, and instructions for the use thereof, and optionally an administration mechanism, e.g., in a single integrated system.

There are numerous optional modes of administering the composition described herein. For example, the composition may be delivered by air-gun delivery or ballistic air delivery.

In certain embodiments, the composition or dosage form can be administered at lease once per week per eye in need of treatment, at least once per day per eye in need of treatment, from 1 to 4 time points per day per eye in need of treatment, or two times per day per eye in need of treatment. In some embodiments, at each time point the composition is administered in an amount from about 1 μl to about 250 μl, about 1 μl to about 200 μl, about 1 μl to about 100 μl, about 2 μl to about 250 μl, about 2 μl to about 200 μl, about 2 μl to about 100 μl, about 3 μl to about 250 μl, about 3 μl to about 200 μl, about 3 μl to about 100 μl, about 4 μl to about 250 μl, about 4 μl to about 200 μl, about 4 μl to about 100 μl, or about 5 μl to about 250 μl, about 5 μl to about 200 μl, or about 5 μl to about 100 μl.

The treatment period can depend on a number of factors, such as the severity of the condition, the age of the subject at diagnosis, or the like. In some embodiments, the composition is administered for a treatment period of at least about 1 week. In further embodiments, the composition is administered for a treatment period of from about 1 week to about 1 year. In still further embodiments, the composition is administered for a treatment period of from about 1 week to about 9 months, or about 1 week or about 26 weeks. In still further embodiments, the composition is administered for a treatment period of about 16 weeks.

In some embodiments, the ophthalmic composition can be administered as an ophthalmic drop. Alternatively, the ophthalmic composition can be administered as a subconjuntival injection. In alternative embodiments, the ophthalmic composition can be administered as a sub-tenon's injection. In further alternative embodiments, the ophthalmic composition can be administered in the form of a topical film, topical gel, contact lens, punctal plug, or the like. In some embodiments, the topical film, topical gel, contact lens, punctal plug, or the like can be configured to biodegrade over time to provide controlled and sustained release of the copper-containing agent.

Keratoconus is manifested by progressive thinning and ectasia which may induce irregular astigmatism resulting in visual impairment and corneal blindness if not treated.

In certain embodiments, the compositions and methods described herein treat astigmatism that is not caused by or associated with keratoconus.

In certain embodiments, the astigmatism is irregular astigmatism.

In further embodiments, the astigmatism is focal astigmatism (e.g., round astigmatism, oval astigmatism).

The astigmatism may be symmetric or asymmetric astigmatism. The astigmatism may also be bow tie astigmatism. The methods of the invention may be customized to match the patient's optical needs, targeting delivery of the copper-containing composition to treat, e.g., focal or bowtie astigmatism, or symmetric or asymmetric astigmatism.

The methods disclosed herein are useful for treating astigmatism and its associated symptoms. The methods of the present invention can thus stabilize, improve, or correct a patient's distorted or blurred vision, alleviate eye strain, diminish a patient's squinting, diminish fatigue, and/or improve night vision. In severe cases of astigmatism, the methods of the present invention may slow vision loss. The methods of the invention may reduce or prevent headaches or migraines associated with astigmatism.

### Pharmaceutical Compositions

In certain embodiments, the copper-containing agent used in the methods of the invention will be formulated in a pharmaceutical composition. For example, the pharmaceutical composition may comprise one or more copper-containing agents and a pharmaceutically acceptable carrier.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or organic esters.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients.

Further examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Methods of preparing these formulations or compositions include the step of bringing into association an active compound, such as a compound of the invention, with the carrier and, optionally, one or more accessory ingredients. In general, the formulations are prepared by uniformly and intimately bringing into association an active with one or more liquid carriers.

Suspensions, in addition to the active compound(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, the route of administration, the time of administration, the rate of clearance or excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In certain embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). Treating may also encompass eliminating the unwanted condition or side effect. As used herein, treating a disease, disorder, or condition includes treating complication(s) of the disease, disorder, or condition, such as by treating the underlying pathophysiology specific to the complication(s) of the disease, disorder, or condition. The subject to whom the therapeutic agent is administered may be asymptomatic or symptomatic.

### EXAMPLES

### Example 1—Copper-Containing Active Agents can Reduce Corneal Astigmatism

This was a randomized, double-masked, placebo-controlled single-site clinical trial with eligible patients randomly assigned to one of three treatment groups:
Group 1 (IVMED-6Wk) received topical copper sulfate eye drops (0.15 mg/mL copper sulfate pentahydrate) BID for 6 weeks and followed by an additional 20 weeks without treatment (total 26 weeks).
Group 2 (IVMED-16Wk) received topical copper sulfate eye drops BID (0.15 mg/mL copper sulfate pentahydrate) for 16 weeks and followed for an additional 10 weeks without treatment (total 26 weeks).
Group 3 (Placebo) received vehicle eye drops BID for 16 weeks and followed for an additional 10 weeks without treatment (total 26 weeks),

As shown by the data below, corneal astigmatism was reduced in the IVMED-16Wk group as compared to the placebo. The placebo group saw increased astigmatism relative to baseline at Week 16 and Week 26. Specific findings include:
There was a statistically significant difference in the change in mean Corneal Astigmatism from Baseline to Week 16 comparing the IVMED-16Wk Group and the Placebo Group (−0.56 D vs 0.60 D; p=0.0149).
There was a statistically significant difference in the change in mean Corneal Astigmatism from Baseline to Week 26 comparing the IVMED-16Wk Group and the Placebo Group (−0.37 D vs. 0.70 D; p=0.01681).
There were no significant differences within or between treatment groups in corneal astigmatism

Corneal Astigmatism was measured in the spherical equivalent (SE) using the Oculus Pentacam at each visit. At Baseline, mean Corneal Astigmatism (±SD): 5.65 (2.51), 5.60 (2.70), 5.63 (2.96) for Groups 1, 2, and 3, respectively. Summary descriptive statistics at Baseline and each follow-up visit for each treatment group are presented in Table 1.

TABLE 1
Corneal Astigmatism (SE): Mean, SD, Median, Min, Max
Corneal Astigmatism (SE)

| | | | | | | | |
|---|---|---|---|---|---|---|---|
| BL ^{A} | Wk 1 | Wk 3 | Wk 6 | Wk 12 | Wk 16 | Wk 21 | Wk 26 |

Group 1 (IVMED-6Wk)

| | | | | | | | | |
|---|---|---|---|---|---|---|---|---|
| Mean | 5.65 | 5.71 | 6.29 | 5.81 | 5.50 | 5.85 | 5.34 | 5.66 |
| SD | 2.51 | 2.62 | 2.21 | 2.60 | 2.66 | 2.90 | 2.33 | 2.89 |
| Median | 5.00 | 5.10 | 5.00 | 4.95 | 5.00 | 4.85 | 5.10 | 4.80 |
| Min, | 2.20, | 2.0, | 4.3, | 3.3, | 2.2, | 1.9. | 2.6. | 1.6, |
| Max | 10.55 | 10.0 | 10.2 | 10.9 | 9.8 | 10.1 | 8.8 | 10.2 |
| N | 9 | 9 | 7 | 8 | 7 | 8 | 5 | 8 |

Group 2 (IVMED-16Wk)

| | | | | | | | | |
|---|---|---|---|---|---|---|---|---|
| Mean | 5.60 | 5.56 | 5.71 | 5.04 | 4.94 | 5.09 | 5.57 | 5.25 |
| SD | 2.70 | 2.72 | 2.68 | 2.29 | 2.34 | 2.23 | 3.56 | 2.68 |
| Median | 5.75 | 5.70 | 6.00 | 5.20 | 4.80 | 5.15 | 4.90 | 4.90 |
| Min, | 1.75, | 1.0, | 0.7, | 1.3, | 1.2, | 1.0, | 1.0, | 1.0, |
| Max | 10.4 | 9.3 | 9.6 | 9.0 | 8.4 | 8.6 | 13.1 | 8.7 |
| N | 14 | 13 | 13 | 11 | 11 | 12 | 9 | 10 |

Group 3 (Placebo)

| | | | | | | | | |
|---|---|---|---|---|---|---|---|---|
| Mean | 5.63 | 6.42 | 6.18 | 6.01 | 5.81 | 6.26 | 6.06 | 6.27 |
| SD | 2.96 | 3.07 | 3.29 | 3.60 | 3.42 | 3.53 | 3.40 | 3.53 |
| Median | 5.25 | 6.25 | 5.90 | 5.35 | 4.90 | 5.65 | 5.80 | 5.85 |
| Min, | 0.75, | 0.9, | 1.5, | 0.4, | 1.2, | 1.3, | 0.8, | 1.3, |
| Max | 10.75 | 11.6 | 12.2 | 12.3 | 11.9 | 12.8 | 11.6 | 12.2 |
| N | 13 | 12 | 12 | 12 | 12 | 12 | 12 | 12 |

A = Baseline (BL) was the mean of the Screening Visit and Day 0, if both visits occurred.

Changes in Corneal Astigmatism from Baseline to Weeks 6, 16, and 26 are presented in Table 2 for each treatment groups. There were statistically significant differences in the changes to Corneal Astigmatism relative to Baseline comparing:
IVMED-16Wk with Placebo at 16 weeks (p=0.0149 without imputation)
IVMED-16Wk vs Placebo at 26 weeks (p=0.01681 with imputation)
   There was a statistically significant trend at Week 26 comparing IVMED-16Wk and Placebo (p=0.05488 without imputation).

TABLE 2
Corneal Astigmatism (SE): Changes from Baseline to Weeks 6, 16 and 26
Corneal Astigmatism (SE): Changes from Baseline to Weeks 6, 16 and 26

| | Change from Baseline | | | |
|---|---|---|---|---|
| Baseline ^{A} | | Week 6 | Week 16 | Week 26 |

Group 1 (IVMED-6 Wk)

| | | | | | |
|---|---|---|---|---|---|
| Mean | 5.65 | Change in | −0.27 | 0.11 | −0.08 |
| | | Mean | | | |
| SD | 2.51 | SD | 1.14 | 1.11 | 1.16 |
| Median | 5.00 | Median | −0.28 | −0.33 | −0.35 |
| Min, Max | 2.20, 10.55 | Min, Max | −1.70, 1.90 | −0.70, 2.70 | −1.30, 2.50 |
| N | 9 | N | 8 | 8 | 8 |

Group 2 (IVMED-16 Wk)

| | | | | | |
|---|---|---|---|---|---|
| Mean | 5.60 | Change in | −0.19 | −0.56 ^{B} | −0.38 ^{C, D} |
| | | Mean | | | |
| SD | 2.70 | SD | 0.55 | 1.08 | 1.23 |
| Median | 5.75 | Median | −0.35 | −0.10 | −0.22 |
| Min, Max | 1.75, 10.40 | Min, Max | −1.10, 0.75 | −3.30, 0.70 | −2.60, 1.60 |
| N | 14 | N | 11 | 12 | 10 |

Group 3 (Placebo)

| | | | | | |
|---|---|---|---|---|---|
| Mean | 5.63 | Change in | 0.35 | 0.60 | 0.62 |
| | | Mean | | | |
| SD | 2.96 | SD | 1.17 | 1.07 | 1.02 |
| Median | 5.25 | Median | 0.03 | 0.47 | 0.57 |
| Min, Max | 0.75, 10.75 | Min, Max | −1.20, 3.10 | −1.20, 2.90 | −0.80, 3.10 |
| N | 13 | N | 12 | 12 | 12 |

A = Baseline (BL) was the mean of the Screening Visit and Day 0, if both visits occurred.
B = Week 16: IVMED-16 Wk vs Placebo; p = 0.0149
C = Week 26: IVMED-16 Wk vs Placebo, p = 0.01681 with imputation
D = Week 26: IVMED-16 Wk vs Placebo, p = 0.05488

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating corneal astigmatism, comprising administering a composition comprising a copper-containing agent to an eye of a subject in need thereof.

2. The method of claim 1, wherein the copper-containing agent is selected from: copper sulfate, copper carbonate, copper acetate, copper chloride, copper gluconate, copper bromide, copper fluoride, copper nitrate, copper iodide, copper perchlorate, copper periodate, copper perbromate, copper permanganate, hemocyanin, copper molybdate, copper thiocyanate, copper tartrate, copper tetrafluoroborate, copper selenide, copper pyrophosphate, GHK-copper, tetra-amine copper sulfate, copper-histidine, and copper-glycinate.

3. (canceled)

4. The method of claim 2, wherein the copper-containing agent is copper sulfate.

5. The method of claim 1, wherein the composition comprises from about 0.001 wt % to about 0.1 wt % of the copper-containing agent.

6. (canceled)

7. The method of claim 1, wherein the composition comprises from about 0.01 mg/mL to about 1.0 mg/mL of the copper-containing agent.

8. (canceled)

9. The method of claim 7, wherein the composition comprises from about 0.1 mg/mL to about 0.2 mg/mL of the copper-containing agent.

10. The method of claim 1, wherein the composition further comprises an excipient selected from: a tonicity agent, a solubilizing agent, a thickener, a polymer, a buffer, a pH adjuster, a preservative, and water.

11. The method of claim 1, wherein the composition does not comprise an additional active ingredient.

12. The method of claim 1, wherein the composition has a tonicity of from about 270 mOsm/kg to about 600 mOsm/kg.

13. The method of claim 1, wherein the composition has a pH of from about 5.0 to about 7.8.

14. (canceled)

15. The method of claim 1, wherein the composition is an ophthalmic composition.

16. The method of claim 15, wherein the composition is an ointment, gel, thin film or eye drop composition.

17. The method of claim 15, wherein the composition is delivered by air-gun delivery or ballistic air delivery.

18. The method of claim 1, wherein the composition is administered at least once per week per eye in need thereof.

19-20. (canceled)

21. The method of claim 18, wherein the composition is administered 2 times per day per eye in need thereof.

22. The method of claim 18, wherein from about 5 μl to about 100 μl of the composition is administered at each time point.

23. The method of claim 1, wherein the composition is administered for a treatment period of at least about 1 week.

24-26. (canceled)

27. The method of claim 23, wherein the composition is administered for a treatment period of about 16 weeks.

28. The method of claim 1, wherein the astigmatism is not caused by or associated with keratoconus.

29. The method of claim 1, wherein the astigmatism is irregular astigmatism, focal astigmatism (e.g., round astigmatism, oval astigmatism), symmetric astigmatism, asymmetric astigmatism or bow tie astigmatism.

30-33. (canceled)
